(19) United States
(12) Patent Application Publication
(10) Pub. No.: US 2007/0037180 A1
(43) Pub. Date: Feb. 15, 2007

(54) Site-specific immunization in order to establish antibodies with specificity for the E7 oncoprotein of high-risk HPV's

(76) Inventors: Olle Nilsson, Goteborg (SE); Christian Fermer, Vastra Frolunda (SE)

Correspondence Address: GAUTHIER & CONNORS, LLP, 225 FRANKLIN STREET, SUITE 2300, BOSTON, MA, 02110, US

(21) Appl. No.: 11/350,453

(22) Filed: Feb. 9, 2006

Related U.S. Application Data
(60) Provisional application No. 60/651,800, filed on Feb. 10, 2005.

(51) Int. Cl. C12Q 1/68; G01N 33/53

(52) U.S. Cl. 435/6; 435/7.1

(57) ABSTRACT

The present invention relates to a method for establishment of antibodies with specificity for high-risk HPVs compared to immunization using the whole antigen of high-risk HPVs, wherein the method includes an exclusion of evolutionary conserved motifs as well as regions with high similarity to E7 proteins of low-risk HPVs, as well as thus obtained antibodies.

### FIELD OF THE INVENTION

The present invention relates to a method for establishment of antibodies specific for predefined regions of the E7 oncoprotein of high-risk human papilloma virus (HPV) strains, which are associated with cervical intraepithelial neoplasia and cervical carcinoma. According to the invention defined parts of the antigens are displayed in fusion with phage coat proteins displayed on the surface of the phage. These phage particles are used as immunogens in order to raise specific antibody responses against the predefined regions. The invention provides an improved method for establishment of monoclonal antibodies (MAbs), polyclonal antibodies (PAbs) or antibody fragments against specific regions of the E7 protein of high-risk HPVs by excluding evolutionary conserved motifs and segments with high similarity to E7 protein of low-risk HPVs, as compared to conventional immunization with intact the antigen.

By excluding evolutionary conserved motifs as well as regions with high similarity to E7 proteins of low-risk HPVs, the invention provides an improved method for establishment of antibodies with specificity for high-risk HPVs compared with immunization with the whole antigen.

### BACKGROUND OF THE INVENTION

Phage display is a technique that allows the expression of foreign peptides or proteins on the surface of phage particles. Since the invention of the technique [1] a large number of different molecules such as hormones, toxins, receptors, antibody fragments, peptide libraries, cDNA libraries, and genomic libraries have been successfully displayed (reviewed in Smith and Petrenko [2]). One of the most successful applications of phage display is the construction of large antibody libraries from which specific binders are selected by biopanning [3].

It has also been demonstrated that antigens presented by filamentous phage efficiently trigger an immune response when used for immunization. This was first demonstrated in mice and rabbits immunized with phage displaying repeat regions of the Plasmodium falciparum circumsporozite protein [4]. Further studies have demonstrated that mice immunized with recombinant phage can acquire immunity towards the pathogen [5-8]. Several studies have shown that phage displaying mimotopes can activate the immune system towards the original antigen [9-12] and that filamentous phage can induce specific cytotoxic T lymphocytes [13, 14].

### SUMMARY OF THE INVENTION

The present invention relates to display of defined segments of the E7 oncoprotein specific for high-risk human HPV strains on phage for use as immunogens in order to establish antibodies of different formats e.g. monoclonal, polyclonal antibodies, single-chain Fvs or Fab fragments. Careful analysis of HPV E7 sequences of both high and low risk HPV strains as well as other viral proteins revealed segments that were specific for the high-risk group of HPVs. The N-terminal of HPV 16 E7 protein (SEQ ID NO: 29) contains two conserved domains (cd1 and cd2) that demonstrate sequence homology to the adenovirus E1A protein (SEQ ID NO: 30) as well as to the simian virus40 (SV40) tumor antigen (SEQ ID NO: 31) (FIG. 1). These conserved domains are necessary for the transforming capability of each of these viral oncoproteins and they are involved in the interaction with a number of critical cellular regulatory proteins including the retinoblastoma tumor suppressor gene product pRB [15]. In addition the conserved motif L×C×E in cd2 is also present in the N-terminal of Cyclin D1 (SEQ ID NO: 32) (FIG. 1). Thus, immunization with the entire E7 protein will most likely result in antibodies with cross-reactivity to these proteins. Sequence alignments of E7 proteins revealed parts of the sequence that differ substantially between the low-risk and the high risk HPVs. However, the diversity is also large between some of the high-risk strains indicating that a reagent for detection of all high-risk strains, using the E7 as target, will have to be composed of antibodies established from a series of immunizations with antigens specific for a group of related strains. In the present investigation, regions of the E7 protein were identified that differ extensively between the low-risk strains and the E7 of HPV 16 and HPV 18 respectively (FIG. 2; SEQ ID NOS 33-52 disclosed respectively in order of appearance). The corresponding gene fragments were cloned into the phage display vectors f88-4 and pMK101 and the expressed peptides were displayed in fusion with the phage coat protein pVIII on the surface of phage particles. The recombinant phage clones were used to immunize mice in order to raise an immune response specific for high-risk HPVs. Thus, by careful sequence analysis and by cloning selected gene fragments into phage vector f88-4, antibodies with the desired specificity for high-risk HPVs could be raised.

### EXAMPLE 1

Cloning and Expression of the HPV 16 and 18 E7 Oncoprotein

1.1. PCR Amplification Complementary DNA (First-strand cDNA synthesis kit, GE Healthcare) prepared from mRNA (Quick prep micro mRNA purification kit, GE Healthcare) from cell lines Ca Ski (ATCC number CRL-1550) and HeLa (ATCC number CCL-2) was used as template for amplification of the coding region of the E7 genes of HPV 16 and 18 respectively using the primers listed in table 1. Approximately 100 ng of each cDNA, was used as template in a reaction mixture containing 0.5 μM of each primer, 75 mM Tris-HCl (pH 8.8 at 25° C.), 20 mM (NH₄)₂SO₄, 0.1% (v/v) Tween 20, 1.5 mM MgCl₂, 0.02 u/μl Taq-polymerase (Abgene) and 0.1 mM of each deoxynucleotide in a final volume of 50 μl with the following temperature cycle repeated 30 times: 1 minute incubations at 95° C., 55° C. and 72° C. The size and purity of the PCR products were checked by separation of 5 μl PCR product on 1% agarose gel. PCR products were purified from primers and deoxynucleotides using QIA quick PCR purification kit (Qiagen) according to the manufacturers instruction.

1.2. Cloning in Expression Vector

Purified PCR products and the expression vector pGEX-6P-3 (GE Helthcare) were digested with the restriction enzymes BamHI and EcoRI (GE Helthcare) and precipitated and ligated together according to standard procedures. The HPV 16 and 18 E7 transcripts were inserted in fusion with glutathione-5-transferase (GST) and the ligated DNA was transformed into E. coli BL21(DE3)pLysS (Promega). Plasmid DNA was prepared using Wizard Plus Minipreps DNA Purification System (Promega) and sequence verified clones were used for protein expression.

1.3. Expression of Recombinant E7 Protein

Clones expressing the E7 oncoprotein of HPV 16 and 18 respectively, grown to OD600=0.8 were induced for 3 hours by the addition of isopropyl β-D-thiogalactoside (IPTG) to a final concentration of 0.1 M. Harvested bacteria were disrupted with lysozyme and the supernatant filtered through 0.2 μm sterile filter (Millex GV). The recombinant E7-GST fusion proteins were bound to glutathione sepharose (Amersham Biosciences), washed and eluted with glutathione elution buffer (Amersham Biosciences). The purity of the E7-GST fusion proteins was tested by NuPAGE Bis-Tris gel electrophoresis (Invitrogen).

### EXAMPLE 2

Cloning of Segments of HPV 16 and 18 E7 Oncoprotein

2.1 PCR Amplification

Two segments of the HPV 16 E7 protein (16-1 and 16-2) and one of HPV 18 were amplified with primers listed in Table 2. Approximately 10 ng of each cDNA, cloned into the expression vector pGEX-6P-3, was used as template in a reaction mixture containing 0.5 μM of each primer, 75 mM Tris-HCl (pH 8.8 at 25° C.), 20 mM (NH₄)₂SO₄, 0.1% (v/v) Tween 20, 1.5 mM MgCl₂, 0.02 u/μl Taq-polymerase (Abgene,) and 0.1 mM of each deoxynucleotide in a final volume of 50 μl with the following temperature cycle repeated 30 times: 1 minute incubations at 95° C., 55° C. and 72° C. The size and purity of the PCR products were checked by separation of 5 μl PCR product on 2% agarose gel. PCR products were purified from primers and deoxynucleotides using QIA quick PCR purification kit (Qiagen) according to the manufacturers instruction.

2.2 Cloning into f88-4

Approximately 100 ng of each PCR product and 5 μg of the phage display cloning vector f88-4 (gift from G P Smith) were digested with of HindIII and PstI (GE Healthcare) according to the manufacturers protocol. The digested cloning vectors were separated on 0.8% agarose gel, the vector band was excised and the DNA purified using the gel extraction kit QIAEX II from Qiagen and approximately 100 ng of purified vector DNA was added to each reaction with digested PCR product. The reactions were extracted with phenol and chloroform and precipitated with sodium acetate and ethanol according to standard procedures. The vector and PCR product pellets were resolved in 20 μl 1×RX buffer and 0.05 units T4 DNA ligase/μl (USB) and ligations were performed at room temperature over night. Of each ligation, 10 μl were transformed into CaCl₂ competent E. coli JM109, mixed in top agar and spread on LB plates supplemented with 40 μg/ml tetracycline and individual clones were picked after incubation at 37° C. for 16-18 hour. Individual clones were grown in 1 ml LB supplemented with 40 μg/ml tetracycline over night and single stranded phage DNA was prepared according to the manual from the phage display peptide library kits of New England Biolabs. Clones were sequenced using ABI Prism BigDye Terminator Cycle Sequencing (Applied Biosystems). Samples were run on an ABI Prism 310.

### EXAMPLE 3

Immunisation with Phage Displaying Segments of Oncoprotein E7

3.1 Production of Phage for Immunization

Each clone was grown in 5 ml LB with 20 μg/ml tetracycline for 8 hours at 37° C. after which the culture was used to inoculate 500 ml LB, 20 μg/ml tetracycline, 1 mM IPTG. After over night growth at 37° C., bacteria were removed by centrifugation at 14500×g at 4° C. for 20 minutes and phage particles were precipitated by the addition of 100 ml 20% PEG 8000, 2.5 ml NaCl PEG. After incubating the tubes for 4-hours on ice, phage particle were recovered by an additional centrifugation step as above. After resolving the phage in 20 ml TBS, remaining bacteria were removed by centrifugation. The phage particles were again precipitated by the addition of 4 ml PEG, NaCl and recovered by a 30 min centrifugation at 11500×g at 4° C. for 30 minutes. After resolving phage particles in 2 ml TBS, remaining bacteria were removed by centrifugation and finally the phage solution was filter sterilized (0.22 μm). Phage preparations were kept in 4° C. until further use. In order to determine the titer of the phage stocks, the absorbance at 269 and 320 nm were determined and used to calculate the titer according to the algorithm by Smith (http://www.biosci.missouri.edu/smithgp/PhageDisplayWebsite/PhageDisplayWebsiteIndex.html).

2.1 Immunization of Mice

BALB/c mice were immunized intra-peritoneal 6 times with 3 to 5 weeks intervals with 5×10¹¹ phage particles mixed with 100 μl MPL+TDM emulsion (Corixa). The total volume varied between 200 and 300 μl. Blood samples were drawn at day 0, in connection with immunizations and finally five days after the last booster. Blood samples were analyzed for anti-E7 antibodies in an ELISA assay. Serial dilutions of the blood samples in Blocker casein in PBS (Pierce) were added to Reacti-Bind glutathione coated wells (Pierce) coated with recombinant HPV 16 E7 and HPV 18 E7, fused to glutathione-5-transferase (GST). Serial dilutions of pre-immune and immune sera in Blocker-casein in PBS were added to coated wells. After one-hour incubation, wells were washed six times (EIA kit wash, CanAg Diagnostics AB) and anti-E7 antibodies were traced by the HRP conjugated rabbit anti mouse antibody from Dakocytomation for another hour. After an additional washing step, Horseradish peroxidase substrate Enhanced K-Blue (Neogen Corporation) was added for detection of binding and plates were analyzed after 30 minutes by measuring absorbance at 620 nm (Vmax, Molecular Corporation). Serum samples from two of three mice immunized with the HPV 18 E7 clone demonstrated low but specific reactivity against recombinant HPV 18 E7 protein. Furthermore, two mice out of three immunized with phage clones displaying fragment 1 of HPV 16 E7 (FIG. 2) demonstrated serum samples with specific antibodies against recombinant HPV 16 E7 protein (FIG. 3). Serum samples from one mouse showed high reactivity even when diluted 1:4000. Thus, immunization with phage particles with defined parts of the E7 protein induced an E7 specific response in mice.

### EXAMPLE 4

Establishment of Monoclonal Antibodies

4.1 Fusion and Cloning

Splenocytes from the two mice, demonstrating moderate and high anti-HPV 16 E7 titers respectively, were fused with myeloma cells P3-X63-Ag8 essentially as described by de St. Groth and Scheidegger [16]. Screening for anti-HPV 16 monoclonal antibodies was performed in Maxisorp microtiter plates (Nunc) coated with goat anti mouse IgG antibodies (Jackson Immunoresearch). After incubation with hybridoma supernatants, the plates were washed and HPV 16 E7-GST fusion protein added followed by a second 60 minute incubation. After another washing step bound antigen was traced and detected by a rabbit anti GST antibody (Santa Cruz Biotechnology) followed by a HRP labelled swine anti rabbit antibody (Dakocytomation). Hybridomas positive after the first screening were further in vitro expanded and subjected to a second screening, after which three hybridomas (E716-1, E716-2 and E716-9) producing antibodies with specific reactivity against HPV16 E7 were identified. These hybridomas were cloned and following another screening, five clones of each original hybridoma were selected.

4.2 Isotyping

Determination of isotypes (G1, G2a, G2b, G3, M, A) was performed in an ELISA. MAb's were traced using isotype specific HRP-conjugated Polyclonal Ab's (Zymed) (Table 3).

4.3 Western Blot Analysis

Three clones were tested for reactivity against E7 in protein lysates from human cancer cell lines, HeLa and Ca Ski, in western blot analysis. Cellular proteins were separated under reducing, denaturating conditions with SDS-polyacylamide gel electrophorese (SDS-PAGE), 12% BisTris-gel (Invitrogen), 1×MES buffer with antioxidant (Invitrogen), and transferred to PVDF membrane. To minimize unspecific binding, membranes and antibodies were blocked in blocking buffer. The membranes were incubated with clone medium (approx. 1 μg mAb/ml) for one hour, washed 3 times for 20 minutes with TBS 0.2% Tween and incubated with secondary antibody, HRP conjugated rabbit anti mouse (Dakocytomation) 1:1000 in blocking buffer for one hour. After another wash the signal was detected with ECL (Amersham Biosciences), following the manufacturers recommendations. All three mAbs demonstrated specific binding to the E7 protein (expected size 10.9 kDa), FIG. 4.

4.4 Epitope Mapping

Phage displaying three overlapping parts of fragment 1 of HPV 16 E7 were produced and used to roughly map the epitopes of the monoclonal antibodies. The phage clones were constructed according to 2.2 and 2.3 using primers in table 4, and phage were amplified as in 3.1. The mapping was performed in an ELISA format, capturing the anti-E7 mAbs in maxisorp wells (Nunc) coated with AffiniPure goat anti mouse IgG (Jackson Immunoresearch). The three different recombinant phage particles were added and binding was detected using a rabbit anti M13 antibody (CanAg Diagnostics) and HRP labelled swine anti rabbit (Dakocytomation). After a few minutes incubation with TMB signals were detected. E716-1:2 and E716-9:1 showed reactivity to the displayed fragments 28-46 and 37-53. Thus the corresponding epitopes of these two antibodies are most likely situated between amino acid residues 37 and 46. MAb E716-2:1 demonstrated reactivity only to peptide fragment 37-53 indicating that the epitope is located in the junction between the peptides 28-46 and 47-62.

4.5 Immunocytochemistry (ICC)

Several methods for mounting and fixing HeLa and Ca Ski cells for ICC were tested. The best results were achieved using a zinc-based fixative (modified protocol from Wester et al., [17]). Detection of mAb bound to protein E7 in the cells was made using the Vectastain ABC-kit (Vector Laboratories). Clone E716-1:2 and E716-9:1 gave a specific staining of Ca Ski cytoplasm (HPV 16), but not HeLa cells (HPV 18).

TABLE 1

Primers used to amplify HPV 16 and
18 E7 for cloning in pGEX-6P-3.

| | Sequence, 5′ to 3′ (SEQ ID NOS 13-16, |
|---|---|
| Primer | respectively in order of appearance) |
| Fwd HPV 16 E7 aa 1 | G GGA TCC CAT GGA GAT ACA CCT ACA TTG |
| Rev HPV 16 E7 aa 98 | G GA ATT C TTA TGG TTT CTG AGA ACA GAT GG |
| Fwd HPV 18 E7 aa 1 | G GGA TCC CAT GGA CCT AAG GCA ACA TTG |
| Rev HPV 18 E7 aa 105 | G GA ATT C TTA CTG CTG GGA TGC ACA CCA C |

TABLE 2

Primers used to amplify parts of E7 for
cloning into phage vector f88-4

| | Sequence, 5′ to 3′ (SEQ ID NOS 17-22, |
|---|---|
| Primer | respectively in order of appearance) |
| 16.1 | CATAAGCTTTGCCGAGGAGGAGGATGAAATAG |
| forward | |
| 16.1 | GATCTGCAGACTTGCAACAAAAGGATACAATATTG |
| reverse | |
| 16.2 | CATAAGCTTTGCCAATATTGTAACCTTTTGTTG |
| forward | |
| 16.2 | GATCTGCAGACAGGTCTTCCAAAGTAC |
| reverse | |
| 18 forward | GATAAGCTTTGCCGAGGAAGAAAACGATGAAATAG |
| 18 reverse | GCGCTGCAGACATACACAACATTGTGTGACG |

TABLE 3
Isotyping of selected clones. Marked in yellow are clones that were chosen
for further characterizations.

| CLONE | ISOTYPE |
|---|---|
| E716-1:1 | IgG₁, IgM |
| E716-1:2 | IgG₂ₐ |
| E716-1:3 | IgG₂ₐ |
| E716-1:4 | IgG₁, IgM |
| E716-1:5 | IgG₂ₐ |
| E716-2:1 | IgG₁ |
| E716-2:2 | IgG₁ |
| E716-2:3 | IgG₁ |
| E716-2:4 | IgG₁ |
| E716-2:5 | IgG₁ |
| E716-9:1 | IgG₂ₐ |
| E716-9:2 | IgG₂ₐ |
| E716-9:3 | IgG₂ₐ, IgM |
| E716-9:4 | IgG₂ₐ |
| E716-9:5 | IgM |

TABLE 4

Primers used to amplify parts of fragment
1 of HPV 16 E7 used for epitope mapping.

| | Sequence, 5′ to 3′ (SEQ ID NOS 23-28, |
|---|---|
| Primer | respectively in order of appearance) |
| Fwd aa 28 | CAT A AGC TTT GCC TTA AAT GAC AGC TCA GAG GAG |
| Rev aa 46 | GAT C TGC AGG TTC TGC TTG TCC AGC TGG AC |
| Fwd aa 37 | CAT A AGC TTT GCC GAA ATA GAT GGT CCA GCT G |
| Rev aa 53 | GAT C TGC AGT ATT GTA ATG GGC TCT GTC CG |
| Fwd aa 47 | CAT A AGC TTT GCC CCG GAC AGA GCC CAT TAC AAT ATT G |
| Rev aa 62 | GAT C TGC AGA GTC ACA CTT GCA ACA AAA GGT TAC |

### BRIEF DESCRIPTION OF THE DRAWINGS

FIG. 1 discloses the sequence homology for two conserved domains (cd1 and cd2) of the N-terminal of HPV 16 E7 protein to the adenovirus E1A protein, the simian virus 40 (SV40) tumor antigen and to Cyclin D1

FIG. 2 discloses a sequence alignment of regions of the E7 protein that differ extensively between the low-risk strains and the E7 of HPV 16 (a) and HPV 18 (b) respectively and the corresponding gene fragments cloned into the phage display vectors

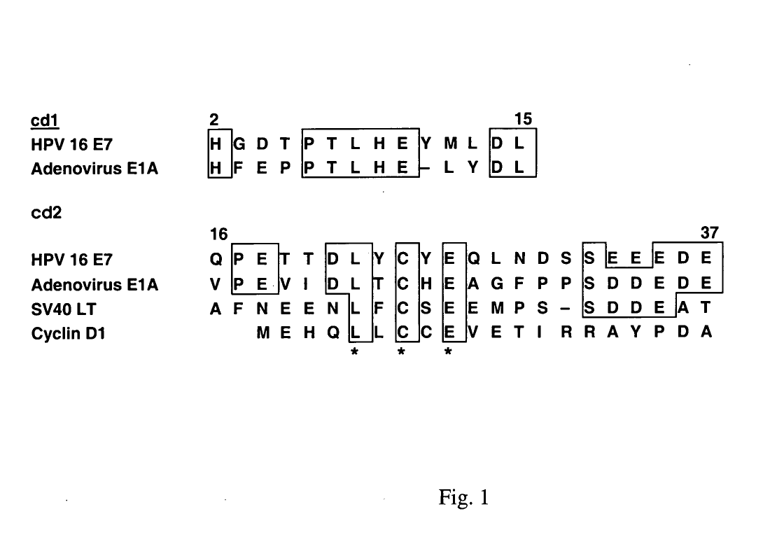

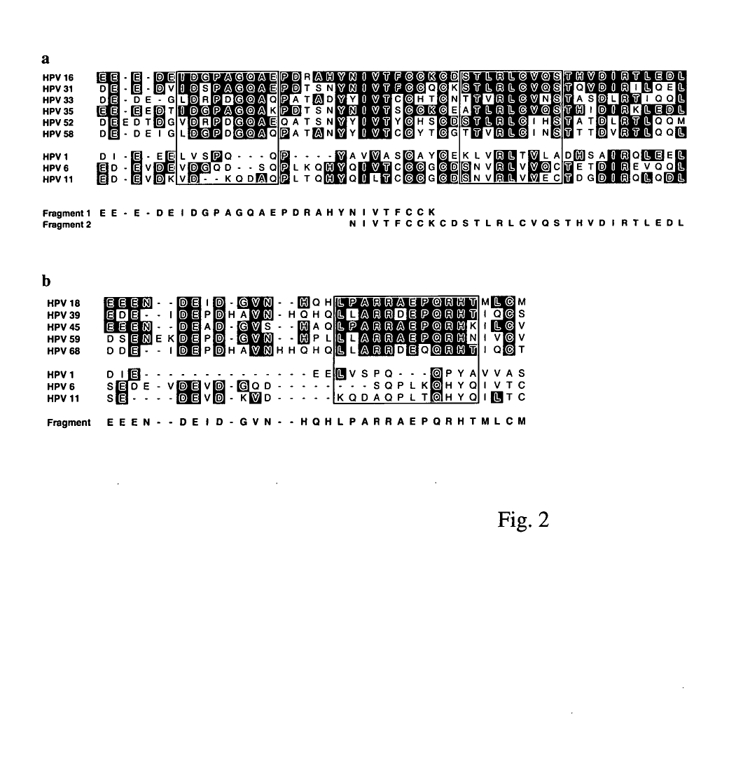

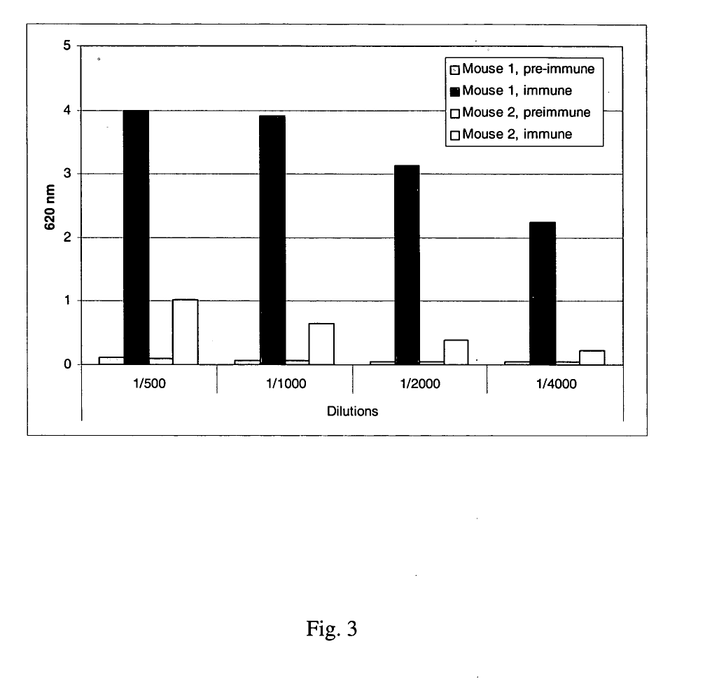

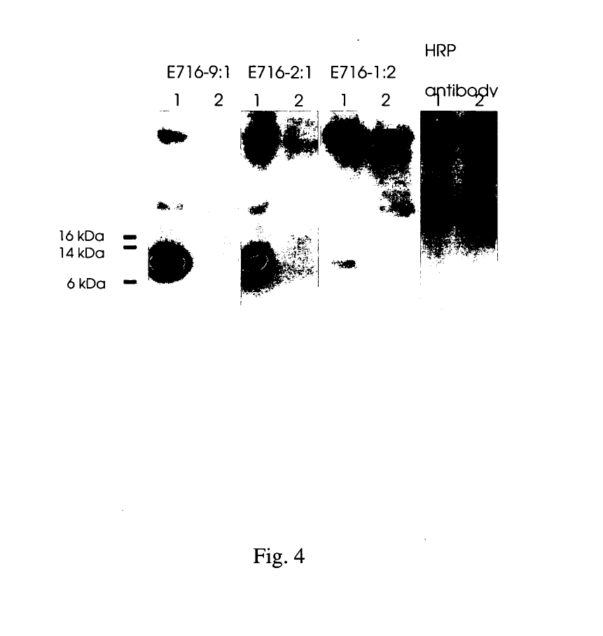

1. A method for establishment of antibodies with specificity for high-risk HPVs compared to immunization using the whole antigen of high-risk HPVs, wherein the method includes an exclusion of evolutionary conserved motifs as well as regions with high similarity to E7 proteins of low-risk HPVs.

2. The method according to claim 1, wherein a fragment of HPV E7.18 high-risk protein EEEN DEID GVN HQH LPARRAEPQRCHTMLCM (SEQ. ID. NO. 7) is used, in particular the fragment part PARRA (SEQ. ID. NO. 10).

3. The method according to claim 1, wherein a fragment of HPV E7.16 high-risk protein EE E DEIDGPAGQAEPDRAHYNIVTFCCK (SEQ. ID. NO. 8) is used, in particular the fragment part NIVTFCCK (SEQ. ID. NO. 11)

4. The method according to claim 1, wherein a fragment of HPV E7.16 high-risk protein NIVTFCCKCDSTLRLCVQSTHVDIRTLEDL (SEQ. ID. NO. 9) is used, in particular the fragment part NIVTFCCK (SEQ. ID. NO. 11) and/or the fragment part LRLCVQST (SEQ. ID. NO. 12).

5. The method according to claim 1, wherein a primer of SEQ. ID. NO. 1 is used to amplify parts of E7 for cloning into phage vector f88-4.

6. The method according to claim 1, wherein a primer of SEQ. ID. NO. 2 is used to amplify parts of E7 for cloning into phage vector f88-4.

7. The method according to claim 1, wherein a primer of SEQ. ID. NO. 3 is used to amplify parts of E7 for cloning into phage vector f88-4.

8. The method according to claim 1, wherein a primer of SEQ. ID. NO. 4 is used to amplify parts of E7 for cloning into phage vector f88-4.

9. The method according to claim 1, wherein a primer of SEQ. ID. NO. 5 is used to amplify parts of E7 for cloning into phage vector f88-4.

10. The method according to claim 1, wherein a primer of SEQ. ID. NO. 6 is used to amplify parts of E7 for cloning into phage vector f88-4.

11. Antibodies with specificity for high-risk HPVs, wherein said antibodies are void of evolutionary conserved motifs as well as regions with high similarity to E7 proteins of low-risk HPVs.


SEQUENCE LISTING (52 sequences)

SEQ ID NO: 1 - DNA, 32 bp, Artificial Sequence
  Description of Artificial Sequence Synthetic primer
cataagctttgccgaggaggaggatgaaatag

SEQ ID NO: 2 - DNA, 35 bp, Artificial Sequence
  Description of Artificial Sequence Synthetic primer
gatctgcagacttgcaacaaaaggatacaatattg

SEQ ID NO: 3 - DNA, 33 bp, Artificial Sequence
  Description of Artificial Sequence Synthetic primer
cataagctttgccaatattgtaaccttttgttg

SEQ ID NO: 4 - DNA, 27 bp, Artificial Sequence
  Description of Artificial Sequence Synthetic primer
gatctgcagacaggtcttccaaagtac

SEQ ID NO: 5 - DNA, 35 bp, Artificial Sequence
  Description of Artificial Sequence Synthetic primer
gataagctttgccgaggaagaaaacgatgaaatag

SEQ ID NO: 6 - DNA, 31 bp, Artificial Sequence
  Description of Artificial Sequence Synthetic primer
gcgctgcagacatacacaacattgtgtgacg

SEQ ID NO: 7 - protein, 31 aa, Human papillomavirus
EEENDEIDGVNHQHLPARRAEPQRCHTMLCM

SEQ ID NO: 8 - protein, 28 aa, Human papillomavirus
EEEDEIDGPAGQAEPDRAHYNIVTFCCK

SEQ ID NO: 9 - protein, 30 aa, Human papillomavirus
NIVTFCCKCDSTLRLCVQSTHVDIRTLEDL

SEQ ID NO: 10 - protein, 5 aa, Human papillomavirus
PARRA

SEQ ID NO: 11 - protein, 8 aa, Human papillomavirus
NIVTFCCK

SEQ ID NO: 12 - protein, 8 aa, Human papillomavirus
LRLCVQST

SEQ ID NO: 13 - DNA, 28 bp, Artificial Sequence
  Description of Artificial Sequence Synthetic primer
gggatcccatggagatacacctacattg

SEQ ID NO: 14 - DNA, 30 bp, Artificial Sequence
  Description of Artificial Sequence Synthetic primer
ggaattcttatggtttctgagaacagatgg

SEQ ID NO: 15 - DNA, 28 bp, Artificial Sequence
  Description of Artificial Sequence Synthetic primer
gggatcccatggacctaaggcaacattg

SEQ ID NO: 16 - DNA, 29 bp, Artificial Sequence
  Description of Artificial Sequence Synthetic primer
ggaattcttactgctgggatgcacaccac

SEQ ID NO: 17 - DNA, 32 bp, Artificial Sequence; identical to SEQ ID NO: 1
  Description of Artificial Sequence Synthetic primer

SEQ ID NO: 18 - DNA, 35 bp, Artificial Sequence; identical to SEQ ID NO: 2
  Description of Artificial Sequence Synthetic primer

SEQ ID NO: 19 - DNA, 33 bp, Artificial Sequence; identical to SEQ ID NO: 3
  Description of Artificial Sequence Synthetic primer

SEQ ID NO: 20 - DNA, 27 bp, Artificial Sequence
  Description of Artificial Sequence Synthetic primer
gatctgcagacaggtcttccaaagtac

SEQ ID NO: 21 - DNA, 35 bp, Artificial Sequence; identical to SEQ ID NO: 5
  Description of Artificial Sequence Synthetic primer

SEQ ID NO: 22 - DNA, 31 bp, Artificial Sequence; identical to SEQ ID NO: 6
  Description of Artificial Sequence Synthetic primer

SEQ ID NO: 23 - DNA, 34 bp, Artificial Sequence
  Description of Artificial Sequence Synthetic primer
cataagctttgccttaaatgacagctcagaggag

SEQ ID NO: 24 - DNA, 30 bp, Artificial Sequence
  Description of Artificial Sequence Synthetic primer
gatctgcaggttctgcttgtccagctggac

SEQ ID NO: 25 - DNA, 32 bp, Artificial Sequence
  Description of Artificial Sequence Synthetic primer
cataagctttgccgaaatagatggtccagctg

SEQ ID NO: 26 - DNA, 30 bp, Artificial Sequence
  Description of Artificial Sequence Synthetic primer
gatctgcagtattgtaatgggctctgtccg

SEQ ID NO: 27 - DNA, 38 bp, Artificial Sequence
  Description of Artificial Sequence Synthetic primer
cataagctttgccccggacagagcccattacaatattg

SEQ ID NO: 28 - DNA, 34 bp, Artificial Sequence
  Description of Artificial Sequence Synthetic primer
gatctgcagagtcacacttgcaacaaaaggttac

SEQ ID NO: 29 - protein, 36 aa, Human papillomavirus
HGDTPTLHEYMLDLQPETTDLYCYEQLNDSSEEEDE

SEQ ID NO: 30 - protein, 35 aa, Human adenovirus type 1
HFEPPTLHELYDLVPEVIDLTCHEAGFPPSDDEDE

SEQ ID NO: 31 - protein, 21 aa, Simian adenovirus
AFNEENLFCSEEMPSSDDEAT

SEQ ID NO: 32 - protein, 20 aa, Homo sapiens
MEHQLLCCEVETIRRAYPDA

SEQ ID NO: 33 - protein, 50 aa, Human papillomavirus
EEEDEIDGPAGQAEPDRAHYNIVTFCCKCDSTLRLCVQSTHVDIRTLEDL

SEQ ID NO: 34 - protein, 50 aa, Human papillomavirus
DEEDVIDSPAGQAEPDTSNYNIVTFCCQCKSTLRLCVQSTQVDIRILQEL

SEQ ID NO: 35 - protein, 50 aa, Human papillomavirus
DEDEGLDRPDGQAQPATADYYIVTCCHTCNTTVRLCVNSTASDLRTIQQL

SEQ ID NO: 36 - protein, 51 aa, Human papillomavirus
EEEEDTIDGPAGQAKPDTSNYNIVTSCCKCEATLRLCVQSTHIDIRKLEDL

SEQ ID NO: 37 - protein, 52 aa, Human papillomavirus
DEEDTDGVDRPDGQAEQATSNYYIVTYCHSCDSTLRLCIHSTATDLRTLQQM

SEQ ID NO: 38 - protein, 51 aa, Human papillomavirus
DEDEIGLDGPDGQAQPATANYYIVTCCYTCGTTVRLCINSTTTDVRTLQQL

SEQ ID NO: 39 - protein, 43 aa, Human papillomavirus
DIEEELVSPQQPYAVVASCAYCEKLVRLTVLADHSAIRQLEEL

SEQ ID NO: 40 - protein, 49 aa, Human papillomavirus
EDEVDEVDGQDSQPLKQHYQIVTCCCGCDSNVRLVVQCTETDIREVQQL

SEQ ID NO: 41 - protein, 49 aa, Human papillomavirus
EDEVDKVDKQDAQPLTQHYQILTCCCGCDSNVRLVVECTDGDIRQLQDL

SEQ ID NO: 42 - protein, 28 aa, Human papillomavirus
EEEDEIDGPAGQAEPDRAHYNIVTFCCK

SEQ ID NO: 43 - protein, 30 aa, Human papillomavirus; identical to SEQ ID NO: 9

SEQ ID NO: 44 - protein, 30 aa, Human papillomavirus
EEENDEIDGVNHQHLPARRAEPQRHTMLCM

SEQ ID NO: 45 - protein, 32 aa, Human papillomavirus
EDEIDEPDHAVNHQHQLLARRDEPQRHTIQCS

SEQ ID NO: 46 - protein, 30 aa, Human papillomavirus
EEENDEADGVSHAQLPARRAEPQRHKILCV

SEQ ID NO: 47 - protein, 32 aa, Human papillomavirus
DSENEKDEPDGVNHPLLLARRAEPQRHNIVCV

SEQ ID NO: 48 - protein, 33 aa, Human papillomavirus
DDEIDEPDHAVNHHQHQLLARRDEQQRHTIQCT

SEQ ID NO: 49 - protein, 18 aa, Human papillomavirus
DIEEELVSPQQPYAVVAS

SEQ ID NO: 50 - protein, 25 aa, Human papillomavirus
SEDEVDEVDGQDSQPLKQHYQIVTC

SEQ ID NO: 51 - protein, 25 aa, Human papillomavirus
SEDEVDKVDKQDAQPLTQHYQILTC

SEQ ID NO: 52 - protein, 30 aa, Human papillomavirus; identical to SEQ ID NO: 44